(19) United States
(12) Patent Application Publication
(10) Pub. No.: US 2021/0139442 A1
(43) Pub. Date: May 13, 2021

(54) D-GLUCARO-6,3-LACTONE MONOESTER AND A PROCESS FOR THE PREPARATION THEREOF

(71) Applicant: BASF SE, Ludwigshafen am Rhein (DE)

(72) Inventors: Dominik Ohlmann, Ludwigshafen (DE); Abhijeet Roy, Navi Mumbai (IN); Boris Tissberger, Ludwigshafen (DE); Thomas Bodenstein, Ludwigshafen (DE); Rahul Mulay, Navi Mumbai (IN)

(21) Appl. No.: 17/047,893

(22) PCT Filed: Apr. 11, 2019
(86) PCT No.: PCT/EP2019/059176

§ 371 Date: Oct. 15, 2020

(30) Foreign Application Priority Data
Apr. 18, 2018 (EP) 18168018.2

(51) Int. Cl. C07D 307/33

(52) U.S. Cl. CPC C07D 307/33

(57) ABSTRACT

Described herein are D-glucaro-6,3-lactone monoester products and a novel process for the preparation of the same.

### FIELD OF INVENTION

The presently claimed invention relates to a product of monoesterification obtained from a D-glucaro-6,3-lactone and an alcohol and a process for the preparation of the same.

### BACKGROUND OF INVENTION

The saccharic acid or glucaric acid is obtained by oxidizing a sugar such as glucose with nitric acid. The sodium salt of glucaric acid is used in dishwasher detergents. In hard-water the sodium salt of glucaric acid acts as chelating agent for calcium and magnesium ions to make the detergents more efficient. The utility of the sodium salt of glucaric acid has replaced environmentally problematic phosphates in most detergents.

The glucaric acid forms 2 isomers of lactonic acid; (1,4) and (3,6) D-glucaro-lactonic acid. The D-glucaro-6,3-lactonic acid has exceptional stability in aqueous solutions and is not hydrolyzed to corresponding dibasic acid.

[chemical structure]

Ethyl, propyl, butyl and amyl monoesters of D-saccharic acid were synthesized by Zinner et. al. [Chem Ber 1956, 1503]. The esterification of glucaro-3,6-lactonic acid with an activated cation exchanger and alcohols produces the glucaro-3,6-lactonic acid monoester, which are also characterized as tribenzoates and tris-p-nitrobenzoates.

EP 0 526 301 A1 discloses synthesis of octyl, dodecyl, octadecyl, hexyl glucaro-1,4-lactone monoester, however, there is no indication in said document about the glucaro-6,3-lactone monoester.

L. A. Mai [Institute of Chemistry of the Academy of Science of the Latvian SSR, Vol. 31. No. 8, 1961] discloses process for the synthesis of methyl ester of the glucaro-6,3-lactone monoester from the dilactone.

Heslop and Smith [Journal of Chemical Society, 1944, pages 633-636] discloses process for the synthesis of the methyl ester of glucaro-6,3-lactone monoester from the D-glucosaccharo-1,4,3,6-dilactone.

WO 2016/131672 discloses a process for the preparation of diester from dilactones. WO 2016/131672 describes that the monoester is one of the products formed in this reaction. One of the disadvantages in preparation of D-glucaro-6,3-lactone monoester is the formation of the diester as one of the by-products. The existing techniques for selectively obtaining D-glucaro-6,3-lactone monoester is not satisfactory in terms of low yield and purity of the final product. Moreover, the relatively high yield and selectivity of undesirable product such as di-ester and acid render the available techniques unfavorable.

Thus, it was an object of the presently claimed invention to provide a process for selectively preparing D-glucaro-6,3-lactone monoester having a high purity with process conditions which render the invention economical by optimizing them in a manner that the formation of diester, D-glucaro-1,4-lactone monoester and di-lactone, is either minimized or eliminated.

### SUMMARY OF INVENTION

Surprisingly, it has been found that the reaction between D-glucaro-6,3-lactonic acid and an alcohol of general formula (I) in the presence of an acid catalyst results in an economical and highly selective process to produce D-glucaro-6,3-lactone monoester. The optimized process conditions of the presently claimed invention provide a selective and economical process for the preparation of D-glucaro-6,3-lactone monoester with only traces or without formation of diester, D-glucaro-1,4-lactone monoester and di-lactone.

Accordingly, in one aspect, the presently claimed invention is directed to a process for the selective preparation of D-glucaro-6,3-lactone monoester comprising the steps of: (A) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20,
Y denotes O and S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl,

in the presence of at least one mineral acid or at least one Lewis acid or at least one carboxylic acid or at least one sulfonic acid to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

In another aspect, the presently claimed invention is directed to a compound of general formula (II)

[chemical structure]

wherein

R₁ denotes unsubstituted, linear or branched, C₆-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

### DETAILED DESCRIPTION OF THE PRESENTLY CLAIMED INVENTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms “first”, “second”, “third” or “(A)”, “(B)” and “(C)” or “(a)”, “(b)”, “(c)”, “(d)”, “i”, “ii” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the presently claimed invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

Accordingly, a process for preparing D-glucaro-6,3-lactone monoester comprises the steps of: (A) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, in the presence of at least one mineral acid or at least one Lewis acid or at least one carboxylic acid or at least one sulfonic acid to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,

wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

More preferably, a process for preparing D-glucaro-6,3-lactone monoester comprises the steps of:
(A) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)H, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl,

in the presence of at least one Lewis acid or at least one carboxylic acid or at least one sulfonic acid to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

Most preferably, a process for preparing D-glucaro-6,3-lactone monoester comprises the steps of:
(B) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl,

in the presence of at least one Lewis acid or at least one carboxylic acid to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

In particularly preferably, a process for preparing D-glucaro-6,3-lactone monoester comprises the steps of:
(C) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl,

in the presence of at least one Lewis acid to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

In another preferred embodiment, the presently claimed invention is directed to a process for preparing D-glucaro-6,3-lactone monoester comprises the steps of:
(D) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl,

in the presence of Al(CH₃—SO₃)₃ to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

D-glucaro-6,3-lactone (formula III)

[chemical structure]

Oxidation of aldoses, for instance, with bromine-water affects only the aldehyde group, converting it into a carboxyl group. By the term “aldose”, it is referred to a monosaccharide containing only one aldehyde group per molecule. The oxidation products are called aldonic acids, for example D-gluconic acid is obtained from D-glucose. When aldoses are oxidized more strongly, for example with concentrated nitric acid, then the primary alcohol group as well as the aldehyde group are transformed into carboxyl groups. The products are polyhydroxydicarboxylic acids known as aldaric acids.

An example of aldaric acid is the aldaric acid derived from glucose, i.e. D-glucaric acid, also known as saccharic acid. Conventional techniques may be employed for obtaining D-glucaric acid. Such techniques are known to a person skilled in the art. Nevertheless, U.S. Pat. No. 2,472,168 illustrates a method for the preparation of D-glucaric acid from glucose using a platinum catalyst in the presence of oxygen and a base. Other oxidation methods, as disclosed in U.S. Pat. Nos. 6,049,004, 5,599,977, 6,498,269 and 8,669,397, may also be employed.

D-glucaro-6,3-lactone can also be obtained from various available techniques. One such technique is discussed by Chen and Kiely [J. Org. Chem. 1996, 61, 5847-5851], wherein a cation exchange resin is added to a mixture of monopotassium D-glucarate and water. Acid form of cation exchange resin is added further with filtration and concentration carried thereafter. D-glucaro-6,3-lactone is obtained after 2-3 days of crystallization in the form of white solids and used for synthesis of head, tail hydroxylated nylons. Troy et. Al. [J. Org. Chem. 2009, 74, 8373-8376] discloses acidification of calcium D-glucarate tetrahydrate with sulfuric acid in the presence of acetone-water. The acidification step is followed by filtration, reduced pressure operation and concentration steps to finally obtain a concentrated aqueous solution containing solid particles of a mixture of D-glucaric acid, D-glucaro-1,4-lactone, D-glucaro-6,3-lactone and D-glucaro-1,4:6,3-lactone in a fixed ratio. For the purpose of the presently claimed invention, the choice of the D-glucaro-6,3-lactone is not limited to the method used to prepare the same.

A person skilled in the art is aware of such methods and may employ any of the available techniques to obtain the same.

Alcohol of general formula I (R₁—OH)

For the purpose of the presently claimed invention, the alcohol of general formula I is

R₁—OH (I),

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

In a preferred embodiment, R₁ denotes

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl, or
—(CH₂CH₂O)ₙH, wherein n is an integer in the range of 1 to 20 or
—(CH₂)ₙYR₂,
wherein n is an integer in the range of 1 to 20.

Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl;

more preferably R₁ is
unsubstituted, linear or branched, C₁-C₂₀ alkyl or
unsubstituted, linear or branched, C₂-C₂₀ alkenyl or
unsubstituted C₃-C₁₀ cycloalkyl or
unsubstituted C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20, Y is O; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl;

even more preferably R₁ is
unsubstituted, linear or branched, C₁-C₂₀ alkyl or
unsubstituted, linear or branched, C₂-C₂₀ alkenyl or
unsubstituted C₃-C₁₀ cycloalkyl or
—(CH₂CH₂O)ₙH, wherein n is an integer in the range of 1 to 20 or
—(CH₂)ₙYR₂,
wherein n is an integer in the range of 1 to 20, Y is O; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl;

most preferably R₁ is
unsubstituted, linear or branched, C₁-C₂₀ alkyl or
unsubstituted C₃-C₁₀ cycloalkyl or
—(CH₂CH₂O)ₙH, wherein n is an integer in the range of 1 to 20 or
—(CH₂)ₙYR₂,
wherein n is an integer in the range of 1 to 20, Y is O; and
R₂ denotes unsubstituted, linear or branched, C₁-C₂₀ alkyl or
unsubstituted or branched C₃-C₁₀ cycloalkyl;

particular preferably R₁ is
unsubstituted, linear or branched, C₁-C₂₀ alkyl or
—(CH₂CH₂)H, wherein n is an integer in the range of 1 to 20.

Within the context of the presently claimed invention, the term “alkyl”, as used herein, refers to an acylic saturated aliphatic groups, including linear or branched alkyl saturated hydrocarbon radical denoted by a general formula CₙH₂ₙ₊₁ and wherein n is the number of carbon atoms 1, 2, 3, 4 etc.

In a preferred embodiment the unsubstituted, linear or branched, C₁-C₂₀ alkyl refers to a branched or unbranched saturated hydrocarbon group having C₁-C₂₀ carbon atoms, more preferably C₂-C₂₀ carbon atoms, even more preferably C₃-C₂₀ carbon atoms, most preferably C₄-C₂₀ carbon atoms, particularly preferably C₅-C₂₀ carbon atoms, even more particularly preferably C₆-C₂₀ carbon atoms.

In a preferred embodiment, R₁ denotes unsubstituted linear C₁-C₂₀ alkyl which preferably selected from the group consisting of, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl and eicosyl; more preferably selected from the group consisting of hexyl, heptyl, octyl, nonyl, decyl, undecyl, dodecyl, tridecyl, tetradecyl, pentadecyl, hexadecyl, heptadecyl, octadecyl, nonadecyl and eicosyl.

In a preferred embodiment, R₁ denotes unsubstituted branched C₁-C₂₀ alkyl which is selected from the group consisting of, but are not limited to, isopropyl, iso-butyl, neo-pentyl, 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl and iso-eicosyl, more preferably selected from the group consisting of 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl and iso-eicosyl.

In a preferred embodiment, R₁ denotes unsubstituted, linear or branched alkenyl preferably selected from C₂-C₂₀ carbon atoms, more preferably C₃-C₂₀ carbon atoms, even more preferably C₄-C₂₀ carbon atoms, most preferably C₅-C₂₀ carbon atoms, particularly preferably C₆-C₂₀ carbon atoms, having at least one C═C double bond in any position, preferably, 1 to 5 C═C double bonds, more preferably 1 to 4 C═C double bonds, even more preferably 1 to 3 C═C double bonds, most preferably 1 to 2 C═C double bonds, particular preferably 1 C═C double bond, wherein each case, each of the carbon atoms is involved not in more than 1 double bond.

Representative examples of C₂-C₂₀ alkenyl containing at least one double bond include, but are not limited to, 1-propenyl, 1-butenyl, 1-pentenyl, 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl, 2-tetradecenyl, 1-pentadecenyl, 2-pentadecenyl, 1-hexadecenyl, 2-hexadecenyl, 1-heptadecenyl, 2-heptadecenyl, 1-octadecenyl, 2-octadecenyl, 1-nonadecenyl, 2-nonadecenyl, 1-eicosenyl and 2-eicosenyl, more preferably selected from 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 2-undecenyl, 1-dodecenyl, 2-dodecenyl, 1-tridecenyl, 2-tridecenyl, 1-tetradecenyl, 2-tetradecenyl, 1-pentadecenyl, 2-pentadecenyl, 1-hexadecenyl, 2-hexadecenyl, 1-heptadecenyl, 2-heptadecenyl, 1-octadecenyl, 2-octadecenyl, 1-nonadecenyl, 2-nonadecenyl, 1-eicosenyl and 2-eicosenyl.

For the purpose of the presently claimed invention, the representative examples of C₂-C₂₀-alkenyl containing two double bonds include, but are not limited to, 1,4-hexadienyl, 1,3-hexadienyl, 2,5-hexadienyl, 3,5-hexadienyl, 2,4-hexadienyl etc.

For the purpose of the presently claimed invention, the representative examples of C₂-C₂₀-alkenyl containing three double bonds include, but are not limited to, 1,3,5-hexatrienyl, 1,3,6-heptatrienyl, 1,4,7-octatrienyl or 2-methyl-1,3,5hexatrienyl etc.

For the purpose of the presently claimed invention, the representative examples of C₂-C₂₀-alkenyl containing four double bonds include, but are not limited to, 1,3,5,7-octatetraenyl, 1,3,5,8-nonatetraenyl, 1,4,7,10-undecatetraenyl, 2-ethyl-1,3,6,8-nonatetraenyl, 2-ethenyl-1,3,5,8-nonatetraenyl etc., and

For the purpose of the presently claimed invention, the representative examples of C₂-C₂₀-alkenyl containing five double bonds include, but are not limited to, 1,3,5,7,9-decapentaenyl, 1,4,6,8,10-undecapentaenyl, 1,4,6,9,11-dodecapentaenyl etc.

For the purpose of the presently claimed invention, the term unsubstituted or branched C₃-C₁₀ cycloalkyl refers to a monocyclic and bicyclic 3 to 10 membered saturated cycloaliphatic radical.

In a preferred embodiment, R₁ denotes unsubstituted or branched C₃-C₁₀ cycloalkyl is monocyclic and bicyclic preferably selected from C₃-C₁₀, more preferably C₄-C₁₀, most preferably C₅-C₁₀, particular preferably C₆-C₁₀

Representative examples of unsubstituted or branched C₃-C₁₀ monocyclic and bicyclic cycloalkyl include, but are not limited to, cyclopropyl, cyclobutyl, cyclopentyl, cyclohexyl, cycloheptyl, bicyclo[2.2.1]heptyl and bicyclo[3.1.1]heptyl.

The C₃-C₁₀ monocyclic and bicyclic cycloalkyl can be further branched with one or more equal or different alkyl groups such as methyl, ethyl, n-propyl, iso-propyl, n-butyl, iso-butyl, n-pentyl, iso-pentyl, neo-pentyl etc. The representative examples of branched C₃-C₁₀ monocyclic and bicyclic cycloalkyl include, but are not limited to methyl cyclohexyl, dimethyl cyclohexyl etc.

In a preferred embodiment, R₁ denotes unsubstituted or branched C₃-C₁₀ cycloalkenyl refers to a to a monocyclic and bicyclic 3 to 10 membered unsaturated cycloaliphatic radical, more preferably C₄-C₁₀, most preferably C₅-C₁₀, articular preferably C₆-C₁₀, which comprises one or more double bonds. Representative examples of C₃-C₁₀ cycloalkenyl include, but are not limited to, cyclobutenyl, cyclopentenyl, cyclohexenyl, cycloheptenyl, cyclooctenyl, cyclononenyl or cyclodecenyl. These radicals can be branched with one or more equal or different alkyl radical, preferably with methyl, ethyl, n-propyl or iso-propyl. The representative examples of branched C₃-C₁₀ monocyclic and bicyclic cycloalkenyl include, but are not limited to methyl cyclohexenyl, dimethyl cyclohexenyl etc.

Within the context of the present invention, the term alkylene refers to acyclic saturated hydrocarbon chains, which combine different moieties. Representative examples of the alkylene groups include, but are not limited to, —CH₂—CH₂—, —CH₂—CH(CH₃)—, —CH₂—CH(CH₂CH₃)—, —CH₂—CH(n-C₃H₇)—, —CH₂—CH(n-C₄H₉)—, —CH₂—CH(n-C₅H₁₁)—, —CH₂—CH(n-C₆H₁₃)—, —CH₂—CH(n-C₇H₁₅)—, —CH₂—CH(n-C₈H₁₇)—, —CH(CH₃)—CH(CH₃)—, —(CH₂)₃—, —(CH₂)₄—, —(CH₂)₅—, —(CH₂)₆—, —(CH₂)₈—, —(CH₂)₁₀—, —C(CH₃)₂—, —CH₂—C(CH₃)₂—CH₂—, and —CH₂—[C(CH₃)₂]₂—CH₂—. Preferred C₂- C₁₀-alkylene are —CH₂—CH₂—, —CH₂—CH(CH₃)—, —CH₂—CH(CH₂CH₃)—, —CH₂—CH(n-C₃H₇)—, —CH₂—CH(n-C₄H₉)—, —CH₂—CH(n-C₆H₁₃)—, and —(CH₂)₄—.

In a preferred embodiment, R₁ denotes unsubstituted C₁-C₁₀ alkylene C₃-C₁₀ cycloalkyl refers to acyclic saturated hydrocarbon chains, which combine a C₃-C₁₀ cycloalkyl group. Alkylene chains can be branched or linear and are unsubstituted and include as in the case of C₁-C₁₀ alkylene 1 to 10 carbon atoms or as in the case of C₁-C₆ alkylene 1 to 6 carbon atoms. C₃-C₁₀ cycloalkyl refers to a monocyclic and bicyclic 3 to 10 membered saturated cycloaliphatic radical, more preferably C₄-C₁₀, even more preferably C₅-C₁₀, most preferably C₅-C₁₀, particular preferably C₆-C₁₀.

Representative examples of unsubstituted C₁-C₁₀ alkylene C₃-C₁₀ cycloalkyl include, but are not limited to, —CH₂—(C₃H₅), —CH₂—(C₄H₇), —CH₂—(C₅H₉), —CH₂—(C₆H₁₁), —CH₂—(C₇H₁₃), —CH₂—CH₂(C₃H₅), —CH₂—CH₂(C₄H), —CH₂—CH₂(C₅H₉), —CH₂—CH₂(C₆H₁₁), —CH₂—CH(C₇H₁₃)—, —CH₂—CH(C₆H₁₁)—, —CH₂—CH(C₅H₉).

In a preferred embodiment, R₁ denotes unsubstituted C₁-C₁₀ alkylene C₃-C₁₀ cycloalkenyl refers to acyclic saturated hydrocarbon chains, which combine a C₃-C₁₀ cycloalkenyl group. Alkylene chains can be branched or linear and are unsubstituted and include as in the case of C₁-C₁₀ alkylene 1 to 10 carbon atoms or as in the case of C₁-C₆ alkylene 1 to 6 carbon atoms. C₃-C₁₀ cycloalkenyl refers to a monocyclic and bicyclic 3 to 10 membered unsaturated cycloaliphatic radical, more preferably C₄-C₁₀, even more preferably C₅-C₁₀, most preferably C₅-C₁₀, particular preferably C₆-C₁₀ having at least one C═C double bond.

Representative examples of unsubstituted C₁-C₁₀ alkylene C₃-C₁₀ cycloalkenyl include, but are not limited to, —CH₂—(C₃H₃), —CH₂—(C₄H₅), —CH₂—(C₅H₇), —CH₂—(C₆H₉), —CH₂—(C₇H₁₁), —CH₂—CH(C₅H₇), —CH₂—CH₂(C₆H₉), —CH₂—CH(C₇H₁₁), —CH₂—CH(C₇H₉)—, —CH₂—CH(C₆H₇)—, —CH₂—CH(C₅H₅)—.

In a preferred embodiment, R₁ denotes —(CH₂CH₂O)ₙH, wherein n is an integer in the range of 1 to 20, preferably in the range of 2 to 10, more preferably 2 to 7, even more preferably 2 to 6, most preferably 3 to 6 and particular preferably 3 to 5. Representative examples of —(CH₂CH₂O)ₙH include, but are not limited to, —(CH₂O)₂H, —(CH₂CH₂O)₃H, —(CH₂CH₂O)₄H, —(CH₂CH₂O)₅H, —(CH₂CH₂O)₆H, —(CH₂CH₂O)₁₂H, —(CH₂CH₂O)₁₅H etc.

In a preferred embodiment, R₁ denotes —(CH₂)ₙYR₂. Within the context of the presently claimed invention, the term ‘n’ is an integer selected from 1 to 20, preferably from 1 to 15, more preferably from 1 to 10, and particular preferably 1 to 6. Within the context of the presently claimed invention, Y denotes O or S, more preferably Y denotes 0.

In a preferred embodiment, R₂ denotes
unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl;
more preferably, R₂ denotes
unsubstituted, linear or branched, C₈-C₂₀ alkyl or
unsubstituted, linear or branched, C₈-C₂₀ alkenyl or
unsubstituted or branched C₃-C₁₀ cycloalkyl or
unsubstituted or branched C₃-C₁₀ cycloalkenyl;
most preferably R₂ denotes
unsubstituted, linear C₈-C₁₈ alkyl or
unsubstituted, linear C₈-C₁₈ alkenyl or
unsubstituted or branched C₆-C₁₀ cycloalkyl;
particular preferably R₂ denotes
unsubstituted, linear C₁₀-C₁₈ alkyl or
unsubstituted, linear C₁₀-C₁₈ alkenyl.

For the purpose of the presently claimed invention, the term unsubstituted, linear or branched, C₁-C₂₀ alkyl or unsubstituted, linear or branched, C₂-C₂₀ alkenyl or unsubstituted or branched C₃-C₁₀ cycloalkyl or unsubstituted or branched C₃-C₁₀ cycloalkenyl or unsubstituted C₁-C₁₀ alkylene C₃-C₁₀ cycloalkyl or unsubstituted C₁-C₁₀ alkylene C₃-C₁₀ cycloalkenyl have same definitions as stated above.

Acids

In a preferred embodiment, in step (A) the at least one acid is selected from the group consisting of mineral acids, Lewis acids, carboxylic acid and sulfonic acids, more preferably the at least one acid is selected from the group consisting of mineral acids, Lewis acids and sulfonic acids, even more preferably the at least one acid is selected from the group consisting of mineral acids and Lewis acids and most preferably the at least one acid is Lewis acid.

In another preferred embodiment, the mineral acid is selected from the group consisting of sulfuric acid, hydrochloric acid, phosphoric acid, perchloric acid, nitric acid, nitrous acid, sulphurous acid, chloric acid, chlorous acid and hypochlorous acid, more preferably the mineral acid is selected from sulfuric acid and hydrochloric acid, even more preferably the mineral acid is sulfuric acid.

In another preferred embodiment, the Lewis acid is a metal-containing compound selected from the group consisting of

a) AsX₃, GaX₃, BX₃, BX₃.(C₂H₅)₂O, BX₃—S(CH₃)₂, AlX₃, (C₂H₅)₂AlX, SbX₃, SbX₅, SnX₂, MgX₂, MgX₂.O(C₂H₅)₂, ZnX₂, BiX₃, FeX₂, TiX₂, TiX₄, NbX₅, NiX₂, CoX₂, HgX₂, whereby X in each case denotes F, C, Br, SO₃, CF₃—SO₃, CH₃—SO₃, or I;

b) BH₃, B(CH₃)₃, GaH₃, AlH₃, Al(acetate)(OH)₂, Al[OCH(CH₃)₂]₃, Al(OCH₃)₃, Al(OC₂H₅)₃, Al₂O₃, (CH₃)₃Al, Ti[OCH(CH₃)₂]₃Cl, Ti[OCH(CH₃)₂]₄, methylaluminum di-(2,6-di-tert-butyl-4-methylphenoxide), methylaluminum di-(4-brom-2,6-di-tert-butylphenoxide), LiClO₄;

c) Mg(acetate)₂, Zn(acetate)₂, Ni(acetate)₂, Ni(NO₃)₂, Co(acetate)₂, Co(NO₃)₂, Cu(acetate)₂, Cu(NO₃)₂, Li(acetate), Zr(acetylacetonate)₄, Si(acetate)₄, K(acetate), Na(acetate), Cs(acetate), Rb(acetate), Mn(acetate)₂, Fe(acetate)₂, Bi(acetate)₃, Sb(acetate)₃, Sr(acetate)₂, Sn(acetate)₂, Zr(acetate)₂, Ba(acetate)₂, Hg(acetate)₂, Ag(acetate), Tl(acetate)₃, Sc(fluoromethansulfonate)₃, Ln(fluoromethanesulfonate)₃, Ni(fluoromethanesulfonate)₂, Ni(tosylate)₂, Co(fluoromethanesulfonate)₂, Co(tosylate)₂, Cu(fluoromethanesulfonate)₂ and Cu(tosylate)₂; more preferably the Lewis acid is selected form the group consisting of

a) BX₃, BX₃—(C₂H₅)₂O, BX₃—S(CH₃)₂, AlX₃, (C₂H₅)₂AlX, TiX₄ whereby X in each case denotes F, Cl, Br, SO₃, CF₃—SO₃, CH₃—SO₃, or I;

b) BH₃, B(CH₃)₃, AlH₃, Al(acetate)(OH)₂, Al[OCH(CH₃)₂]₃, Al(OCH₃)₃, Al(OC₂H₅)₃, Al₂O₃, (CH₃)₃Al, Ti[OCH(CH₃)₂]₃Cl, Ti[OCH(CH₃)₂]₄, methylaluminum di-(2,6-di-tert-butyl-4-methylphenoxide), methylaluminum di-(4-brom-2,6-di-tert-butylphenoxide);

c) Sc(fluoromethansulfonate)₃, Ln(fluoromethanesulfonate)₃, Ni(fluoromethanesulfonate)₂, Co(fluoromethanesulfonate)₂, Cu(fluoromethanesulfonate)₂;

most preferably the Lewis acid is selected form the group consisting of BX₃, BX₃.S(CH₃)₂, AlX₃, and TiX₄, whereby X in each case denotes F, Cl, CF₃—SO₃, or CH₃—SO₃, particular preferably the Lewis acid is AlX₃, whereby X denotes F, Cl, CF₃—SO₃, or CH₃—SO₃.

In a preferred embodiment, the carboxylic acid is selected from the group consisting of trifluoroacetic acid, difluoroacetic acid, fluoroacetic acid, trichloroacetic acid, dichloroacetic acid, monochloroacetic, tribromoacetic acid, dibromoacetic acid, bromoacetic acid and iodoacetic acid. More preferably, the carboxylic acid is selected from the group consisting of trifluoroacetic acid, difluoroacetic acid, fluoroacetic acid, trichloroacetic acid, dichloroacetic acid, monochloroacetic, tribromoacetic acid, dibromoacetic acid and bromoacetic acid. Even more preferably, the carboxylic acid is selected from the group consisting of trifluoroacetic acid, difluoroacetic acid, fluoroacetic acid, trichloroacetic acid, dichloroacetic acid, monochloroacetic and tribromoacetic acid. Most preferably, the carboxylic acid is selected from the group consisting of trifluoroacetic acid, difluoroacetic acid, trichloroacetic acid and dichloroacetic acid. In particularly preferably, the carboxylic acid is selected from the group consisting of trifluoroacetic acid, difluoroacetic acid and trichloroacetic acid. Even in particularly preferably, the carboxylic acid is trifluoroacetic acid.

In a preferred embodiment, the sulfonic acid is selected from the group consisting of methanesulfonic acid, ethanesulfonic acid, 1-propanesulfonic acid, 1-butanesulfonic acid, trifluoromethanesulfonic acid, benzenesulfonic acid, p-toluenesulfonic acid, p-xylene-2-sulfonic acid, naphathalene-1-sulfonic acid and naphthalene-2-sulfonic acid, more preferably the sulfonic acid is selected from the group consisting of methanesulfonic acid, trifluoromethanesulfonic acid, benzenesulfonic acid and p-toluenesulfonic acid, even more preferably methanesulfonic acid and p-toluenesulfonic acid.

Molecular Sieve

In a preferred embodiment, the process of the presently claimed invention is carried out in the presence of one or more molecular sieves. Where the process of the presently claimed invention is carried out in the presence of one or more molecular sieves, preference is given to using one to three molecular sieves, more preferably one or two molecular sieves, even more preferably one (1) molecular sieve. Examples of suitable molecular sieves are molecular sieves having a pore size in the range from 0.1 to 10 angstroms, preferably 3 to 7 angstroms, more preferably 3 to 6 angstroms, very preferably 3 to 4 angstroms.

In a preferred embodiment of the presently claimed invention, the process of the presently claimed invention is carried out in the presence of a molecular sieve having a pore size of 3 angstroms, and the molecular sieve having a pore size of 3 angstroms and the compound of formula (III) are used in general in a weight ratio of 1:10 to 10:1, preferably of 1:1 to 5:1, more preferably of 1.5:1 to 4:1, very preferably of 2:1 to 3:1.

An advantage of using one or more molecular sieves, more particularly of using a molecular sieve having a pore size of 3 angstroms, is its ability to take up liberated water molecules, and in doing so remove water from the equilibrium.

Polar Aprotic Solvent

In a preferred embodiment, the process is carried out in presence of at least one polar aprotic solvent.

Within the context of the presently claimed invention, the term “polar aprotic solvent” refers to an organic solvent having a dipole moment in the range of 0.2 to 5, more preferably in the range of 0.2 to 3, most preferably in the range of 0.2 to 2 and a water solubility of at least about 5% (volume) at ambient temperature, i.e., about 20° C., and which does not undergo significant hydrogen exchange at approximately neutral pH, i.e., in the range of 5 to 9, or preferably in the range 6 to 8.

In another preferred embodiment, the at least one polar aprotic solvent is selected from the group consisting of ethers, lactones, carbonates, sulfones, N,N-dimethylformamide, N,N-dimethylacetamide, acetonitrile, dimethylsulfoxide, N-methyl-pyrrolidone and N-ethyl-pyrrolidone; more preferably the at least one polar aprotic solvent is selected from the group consisting of ethers, acetonitrile, dimethylsulfoxide, N-methyl-pyrrolidone, even more preferably the at least one polar aprotic solvent is selected from the group consisting of ethers, dimethylsulfoxide, N-methyl-pyrrolidone, most preferably the at least one polar aprotic solvent is an ether.

Within the context of the presently claimed invention, the ether is preferably selected from the group consisting of methyl tert-butyl ether, dioxane, diethoxy methane, dimethoxy methane, tetrahydrofuran and tetrahydropyran, more preferably the ether is selected from tetrahydrofuran and dioxane, even more preferably the ether is dioxane.

The weight ratio between the at least one polar aprotic solvent and the D-glucaro-3,6-lactone is preferably in the range of ≥30:1 to ≤1:1. More preferably, the ratio is in the range of ≥20:1 to ≤1:1, or ≥10:1 to ≤1:1, or ≥8:1 to ≤2:1, or ≥5:1 to ≤2:1, or ≥3:1 to ≤2:1. Even more preferably, it is in the range of ≥20:1 to ≤1:1, or ≥10:1 to ≤1:1, or ≥8:1 to ≤2:1, or ≥5:1 to ≤2:1, or ≥3:1 to ≤2:1. Most preferably, in the range of ≥10:1 to ≤1:1, or ≥8:1 to ≤2:1, or ≥5:1 to ≤2:1.

In a preferred embodiment, the molar ratio between the at least one alcohol of general formula (I) and D-glucaro-6,3-lactone is in the range of ≥0.1:1 to ≤5:1, more preferably in the range of ≥0.5:1 to ≤2:1, even more preferably in the range of ≥0.8:1 to ≤2:1, most preferably 1:1.

In a preferred embodiment, the at least one acid is present in the process in an amount in the range of ≥0.01 mol.-% to ≤20 mol.-%, more preferably in the range of ≥0.01 mol.-% to ≤10 mol.-%, even more preferably in the range of ≥0.01 mol.-% to ≤5 mol.-%, most preferably in the range of ≥0.05 mol.-% to ≤2 mol.-%, particular preferably in the range of ≥0.08 mol.-% to ≤0.5 mol.-%, in each case is in relation to the D-glucaro-6,3-lactone

In another embodiment, the process of the presently claimed invention is carried out at a temperature in the range of ≥30° C. to ≤90° C., more preferably ≥50° C. to ≤80° C., most preferably ≥60° C. to ≤80° C., and particular preferably in the range of ≥65° C. to ≤75° C. The alcohol of general formula (I) and D-glucaro-6,3-lactone form a homogenous mixture upon adding into the polar aprotic solvent. To the thus obtained homogenous mixture the at least one acid catalyst is added. The solution obtained after addition of the at least one acid catalyst is homogenous or heterogenous. This mixture is heated to a temperature in the range of ≥30° C. to ≤90° C. For carrying out the heating to a temperature in the range of ≥30° C. to ≤90° C., any suitable techniques can be used. A person skilled in the art is aware of such techniques.

The compound of formula (II) formed in the reaction is isolated by any method known in the art selected from the group consisting of chemical separation, acid-base neutralization, distillation, evaporation, column chromatography, filtration, concentration, crystallization and re-crystallization or a combination thereof. A person skilled in the art is aware of such techniques.

Accordingly, in an embodiment the compound of general formula (II) as obtained according to the process of the presently claimed invention has a general structure as shown herein below

[chemical structure]

wherein

R₁ denotes unsubstituted, linear or branched, C₆-C₂₀ alkyl or
unsubstituted, linear or branched, C₆-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ is independently selected from
unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

Accordingly, in a preferred embodiment the compound of general formula (II) obtained by the above described process is Decyl-D-glucaro-6,3-lactone monoester (decyl(2R)-2-[(2S,3R,4S)-3,4-dihydroxy-5-oxo-tetrahydrofuran-2-yl]-2-hydroxy-acetate) and is represented as shown below.

[chemical structure]

Accordingly, in a preferred embodiment the compound of general formula (II) obtained by the above described process is Dodecyl-D-glucaro-6,3-lactone monoester [dodecyl(2R)-2-[(2S,3R,4S)-3,4-dihydroxy-5-oxo-tetrahydrofuran-2-yl]-2-hydroxy-acetate] and is represented as shown below.

[chemical structure]

Accordingly, in a preferred embodiment the compound of general formula (II) obtained by the above described process is Tetradecyl-D-glucaro-6,3-lactone monoester [tetradecyl(2R)-2-[(2S,3R,4S)-3,4-dihydroxy-5-oxo-tetrahydrofuran-2-yl]-2-hydroxy-acetate] and is represented as shown below.

[chemical structure]

Accordingly, in a preferred embodiment the compound of general formula (II) obtained by the above described process is Octyl-D-glucaro-6,3-lactone monoester [octyl(2R)-2-[(2S,3R,4S)-3,4-dihydroxy-5-oxo-tetrahydrofuran-2-yl]-2-hydroxy-acetate] and is represented as shown below.

[chemical structure]

Accordingly, in a preferred embodiment the compound of general formula (II) obtained by the above described process is Hexadecyl-D-glucaro-6,3-lactone monoester [hexadecyl(2R)-2-[(2S,3R,4S)-3,4-dihydroxy-5-oxo-tetrahydrofuran-2-yl]-2-hydroxy-acetate] and is represented as shown below.

[chemical structure]

Another aspect of the presently claimed invention relates to the compounds of general formula (II)

[chemical structure]

wherein

R₁ denotes unsubstituted, linear or branched, C₆-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20,
Y denotes O or S; and
R₂ is independently selected from
unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

The preferred embodiments of the inventively claimed process also apply to the inventively claimed compounds.

The presently claimed invention offers one or more of following advantages:

The novel synthesis route, as described hereinabove, has several advantages over the current state of the art. The current state of the art available, such as but not limited to the one described by, Zenner et. al [Institute of Organic Chemistry at the University of Rostock, Mar. 12, 1956] reports to have obtained monoester of D-glucaric acid with nearly 47 mol. equivalents of the alcohol used therein. However, surprisingly the novel synthesis route of the presently claimed process provides D-glucaro-(6,3) lactone monoester even at very low mol. equivalent, such as those described hereinabove, with traces or without formation of the by-products and/or unwanted impurities. Another advantage of the presently claimed invention is that a very low quantity of alcohol is used as well as the use of easily available raw materials, such as but not limited to, the at least one polar aprotic solvent, as described hereinabove.

In the following, specific embodiments of the presently claimed invention are described:

1. A process for preparing D-glucaro-6,3-lactone monoester comprising the steps of:

(A) reacting D-glucaro-6,3-lactone with at least one alcohol of a general formula (I)

R₁—OH (I),

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1-20,
Y denotes O or S; and
R₂ is independently selected from
unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
   unsubstituted C₁-C₁₀ alkylene C₃-C₁₀ cycloalkenyl,

in the presence of at least one mineral acid or at least one Lewis acid or at least one carboxylic acid or at least one sulfonic acid to obtain a compound of general formula (II)

[chemical structure]

wherein

R₁ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1-20,
Y denotes O and S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

2. The process according to embodiment 1, characterized in that unsubstituted, linear C₁-C₂₀ alkyl is selected from the group consisting of methyl, ethyl, propyl, butyl, pentyl, hexyl, heptyl, octyl, nonyl, decyl, undecyl, dodecyl, tridecyl, tetradecyl, pentadecyl, octadecyl, heptadecyl, octadecyl, nonadecyl and eicosyl.

3. The process according to embodiment 1, characterized in that unsubstituted, branched C₁-C₂₀ alkyl is selected from the group consisting of iso-propyl, iso-butyl, t-butyl, iso-pentyl, neo-pentyl, 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl and iso-eicosyl.

4. The process according to embodiment 1, characterized in that R₁ denotes
unsubstituted, linear or branched, C₁-C₂₀ alkyl or
unsubstituted, linear or branched, C₂-C₂₀ alkenyl or
unsubstituted C₃-C₁₀ cycloalkyl or
unsubstituted C₃-C₁₀ cycloalkenyl or
—(CH₂CH₂O)ₙH, wherein n is an integer in the range of 1 to 20 or
—(CH₂)ₙYR₂,
wherein n is an integer in the range of 1 to 20, Y is O; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl.

5. The process according to embodiment 4, characterized in that R₁ denotes
unsubstituted, linear or branched, C₁-C₂₀ alkyl or
unsubstituted, linear or branched, C₂-C₂₀ alkenyl or
unsubstituted C₃-C₁₀ cycloalkyl or
—(CH₂CH₂O)ₙH, wherein n is an integer in the range of 1 to 20 or
—(CH₂)ₙYR₂,
wherein n is an integer in the range of 1 to 20, Y is O; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl.

6. The process according to embodiment 4, characterized in that R₁ denotes
unsubstituted, linear or branched, C₁-C₂₀ alkyl or
unsubstituted C₃-C₁₀ cycloalkyl or
—(CH₂CH₂O)ₙH, wherein n is an integer in the range of 1 to 20 or
—(CH₂)ₙYR₂,
wherein n is an integer in the range of 1 to 20, Y is O; and
R₂ denotes unsubstituted, linear or branched, C₁-C₂₀ alkyl or
unsubstituted or branched C₃-C₁₀ cycloalkyl.

7. The process according to embodiment 4, characterized in that R₁ denotes
unsubstituted, linear or branched, C₁-C₂₀ alkyl or
—(CH₂CH₂O)ₙH, wherein n is an integer in the range of 1 to 20

8. The process according to embodiment 1, characterized in that in step (A) the at least one mineral acid is selected from the group consisting of sulfuric acid, hydrochloric acid, phosphoric acid, perchloric acid, nitric acid, nitrous acid, sulphurous acid, chloric acid, chlorous acid and hypochlorous acid.

9. The process according to embodiment 8, characterized in that in step (A) the at least one mineral acid is sulfuric acid

10. The process according to embodiment 1, characterized in that in step (A) the at least one Lewis acid is a metal-containing compound selected from the group consisting of

a) AsX₃, GaX₃, BX₃, BX₃.(C₂H₅)₂O, BX₃—S(CH₃)₂, AlX₃, (C₂H₅)₂AlX, SbX₃, SbX₅, SnX₂, MgX₂, MgX₂O(C₂H₅)₂, ZnX₂, BiX₃, FeX₂, TiX₂, TiX₄, NbX₅, NiX₂, CoX₂, HgX₂, whereby X in each case denotes F, C, Br, SO₃, CF₃—SO₃, CH₃—SO₃, or I,

b) BH₃, B(CH₃)₃, GaH₃, AlH₃, Al(acetate)(OH)₂, Al[OCH(CH₃)₂]₃, Al(OCH₃)₃, Al(OC₂H₅)₃, Al₂O₃, (CH₃)₃Al, Ti[OCH(CH₃)₂]₃Cl, Ti[OCH(CH₃)₂]₄, methylaluminum di-(2,6-di-tert-butyl-4-methylphenoxide), methylaluminum di-(4-brom-2,6-di-tert-butylphenoxide), LiClO₄,

c) Mg(acetate)₂, Zn(acetate)₂, Ni(acetate)₂, Ni(NO₃)₂, Co(acetate)₂, Co(NO₃)₂, Cu(acetate)₂, Cu(NO₃)₂, Li(acetate), Zr(acetylacetonate)₄, Si(acetate)₄, K(acetate), Na(acetate), Cs(acetate), Rb(acetate), Mn(acetate)₂, Fe(acetate)₂, Bi(acetate)₃, Sb(acetate)₃, Sr(acetate)₂, Sn(acetate)₂, Zr(acetate)₂, Ba(acetate)₂, Hg(acetate)₂, Ag(acetate), Tl(acetate)₃, Sc(fluoromethansulfonate)₃, Ln(fluoromethanesulfonate)₃, Ni(fluoromethanesulfonate)₂, Ni(tosylate)₂, Co(fluoromethanesulfonate)₂, Co(tosylate)₂, Cu(fluoromethanesulfonate)₂ and Cu(tosylate)₂.

11. The process according to embodiment 10, characterized in that the at least one Lewis acid is selected from the group consisting of BX₃, BX₃.S(CH₃)₂, AlX₃, SbX₃, and TiX₄, whereby X in each case denotes F, Cl, CF₃—SO₃, or CH₃—SO₃.

12. The process according to embodiment 10 or 11, characterized in that in step (A) the at least one Lewis acid is Al(CH₃—SO₃)₃.

13. The process according to embodiment 1, characterized in that in step (A) the at least one carboxylic acid is selected from the group consisting of trifluoroacetic acid, difluoroacetic acid, fluoroacetic acid, trichloroacetic acid, dichloroacetic acid, monochloroacetic, tribromoacetic acid, dibromoacetic acid, bromoacetic acid and iodoacetic acid.

14. The process according to embodiment 1, characterized in that in step (A) the at least one sulfonic acid is selected from the group consisting of methanesulfonic acid, ethanesulfonic acid, 1-propanesulfonic acid, 1-butanesulfonic acid, trifluoromethanesulfonic acid, benzenesulfonic acid, p-toluenesulfonic acid, p-xylene-2-sulfonic acid, naphathalene-1-sulfonic acid and naphthalene-2-sulfonic acid.

15. The process according to one or more of embodiments 1 to 14, characterized in that step (A) is carried out in the presence of a molecular sieve.

16. The process according to embodiment 15, characterized in that in step (A) the molecular sieve has a pore diameter in the range of ≥0.1 Å to ≤10 Å.

17. The process according to embodiments 1 to 7, wherein step (A) is carried out in presence of at least one polar aprotic solvent.

18. The process according to embodiment 17, characterized in that the at least one polar aprotic solvent is selected from the group consisting of ethers, lactones, carbonates, sulfones, N,N-dimethylformamide, N,N-dimethylacetamide, acetonitrile, dimethylsulfoxide, N-methyl-pyrrolidone and N-ethyl-pyrrolidone.

19. The process according to embodiment 18, characterized in that the at least one ether is selected from the group consisting of methyl tert-butyl ether, dioxane, diethoxy methane, dimethoxy methane, tetrahydrofuran and tetrahydropyran.

20. The process according to embodiment 19, characterized in that the at least one ether is dioxane.

21. The process according to one or more of embodiments 1 to 20, characterized in that in step (A) the molar ratio between the at least one alcohol of general formula (I) and D-glucaro-6,3-lactone is in the range of ≥0.1:1 to ≤5:1.

22. The process according to one or more of embodiments 1 to 13, characterized in that in step (A) the at least one acid is present in an amount in the range of ≥0.01 mol.-% to ≤20 mol.-% in relation to the D-glucaro-6,3-lactone.

23. The process according to one or more of embodiments 1 to 22, characterized in that in step (A) the molecular sieve is present in an amount in the range of ≥10 wt.-% to ≤40 wt.-% in relation to the of D-glucaro-6,3-lactone.

24. The process according to one or more of embodiments 1 to 23, characterized in that the weight ratio between the at least one polar aprotic solvent and D-glucaro-6,3-lactone is in the range of ≥1:1 to ≤10:1.

25. The process according to one or more of embodiments 1 to 24, characterized in that in step (A) the reaction is carried out by stirring at a rotational speed in the range of ≥100 rpm to ≤500 rpm for a period in the range of ≥1 h to ≤10 h.

26. The process according to one or more of embodiments 1 to 25, characterized that step (A) is carried out at a temperature in the range of ≥30° C. to ≤90° C.

27. A compound of general formula (II)

[chemical structure]

wherein

R₁ denotes unsubstituted, linear or branched, C₆-C₂₀ alkyl or
unsubstituted, linear or branched, C₆-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl or
—(CH₂CH₂O)ₙH, wherein n is an integer in the range of 1 to 20 or
—(CH₂)ₙYR₂,
wherein n is an integer in the range of 1-20,
Y denotes O or S; and
R₂ denotes unsubstituted, linear or branched, C₁-C₂₀ alkyl or
unsubstituted, linear or branched, C₂-C₂₀ alkenyl or
unsubstituted or branched C₃-C₁₀ cycloalkyl or
unsubstituted or branched C₃-C₁₀ cycloalkenyl or
unsubstituted C₁-C₁₀ alkylene C₃-C₁₀ cycloalkyl or
unsubstituted C₁-C₁₀ alkylene C₃-C₁₀ cycloalkenyl.

28. The compound of formula (II) according to embodiment 27 is selected from the group consisting of
1. Decyl(2R)-2-[(2S,3R,4S)-3,4-dihydroxy-5-oxo-tetrahydrofuran-2-yl]-2-hydroxy-acetate
2. Dodecyl(2R)-2-[(2S,3R,4S)-3,4-dihydroxy-5-oxo-tetrahydrofuran-2-yl]-2-hydroxy-acetate
3. Tetradecyl(2R)-2-[(2S,3R,4S)-3,4-dihydroxy-5-oxo-tetrahydrofuran-2-yl]-2-hydroxy-acetate
4. Octyl(2R)-2-[(2S,3R,4S)-3,4-dihydroxy-5-oxo-tetrahydrofuran-2-yl]-2-hydroxy-acetate and
5. Hexadecyl(2R)-2-[(2S,3R,4S)-3,4-dihydroxy-5-oxo-tetrahydrofuran-2-yl]-2-hydroxy-acetate.

While the presently claimed invention has been described in terms of its specific embodiments, certain modifications and equivalents will be apparent to those skilled in the art and are intended to be included within the scope of the presently claimed invention.

### EXAMPLES

Chemicals:
D-glucaro-6,3-lactone
Alcohol of general formula (I): decanol, dodecanol, tetradecanol, octanol and hexadecanol
Polar aprotic solvent: dioxane
Acid catalyst: aluminium methanesulfonate
molecular sieves 3 Å

are available from Sigma Aldrich.

General Procedure for Synthesis of D-Glucaro-6,3-Lactone Monoester:

D-glucaro-6,3-monolactone (96.1% purity, 0.65 mol, 131 g), alcohol (1.0 equiv., 0.65 mol), aluminum methanesulfonate (0.1 equiv., 0.065 mol, 20.4 g), optionally molecular sieves 3 Å (28 g) and dioxane (360 g) were charged to a reaction flask. The mixture was stirred at 50-80° C. until completion of the reaction. The reaction mass was added to 200 mL of 5% aqueous sodium bicarbonate solution and the organic layer was separated. The organic layer was washed with water, dried over anhydrous sodium sulfate and then concentrated to yield crude product. The crude product was isolated by crystallization using a suitable solvent to obtain the desired D-glucaro-6,3-lactone monoester.

### Synthesis of Decyl-D-glucaro-6,3-lactone monoester [decyl(2R)-2-[(2S,3R,4S)-3,4-dihydroxy-5-oxo-tetrahydrofuran-2-yl]-2-hydroxy-acetate]

Above reaction was carried out similarly using decanol (0.65 moles) as alcohol. The product was crystallized from heptane to obtain the desired decyl-D-glucaro-6,3-lactone monoester (29 g).

¹H NMR (500 MHz, DMSO-d₆, TMS): δ 6.18-6.16 (d, 1H), δ 5.89-5.86 (d, 1H), δ 5.47-5.46 (d, 1H), δ 4.51-4.44 (m, 2H), δ 4.26-4.21 (t, 2H), δ 4.06-4.05 (d, 2H), δ 1.58-1.56 (d, 2H), δ 1.25 (bs, 14H), δ 0.85-8.83 (t, 3H).

¹³C NMR (500 MHz, DMSO-d₆, TMS): δ 175.8, δ 170.1, δ 80, δ 70.1, δ 69.6, δ 69.2, δ 64.2, δ 31.2, δ 28.94, δ 28.93, δ 28.68, δ 28.66, δ 27.9, δ 25.2, δ 22, δ 13.8.

### Synthesis of Dodecyl-D-glucaro-6,3-lactone monoester [dodecyl(2R)-2-[(2S,3R,4S)-3,4-dihydroxy-5-oxo-tetrahydrofuran-2-yl]-2-hydroxy-acetate]

Above reaction was carried out similarly using dodecanol (0.05205 moles) as alcohol. The product was crystallized from heptane to obtain the desired dodecyl-D-glucaro-6,3-lactone monoester (1.8 g).

¹H NMR (500 MHz, DMSO-d₆, TMS): δ 6.19-6.17 (d, 1H), δ 5.89-5.87 (d, 1H), δ 5.48-5.46 (d, 1H), δ 4.51-4.43 (m, 2H), δ 4.25-4.18 (t, 2H), δ 4.08-4.03 (m, 2H), δ 1.60-1.56 (t, 2H), δ 1.24 (bs, 18H), δ 0.87-8.83 (t, 3H).

¹³C NMR (500 MHz, DMSO-d₆, TMS): δ 175.8, δ 170.1, δ 80, δ 70.1, δ 69.6, δ 69.2, δ 64.2, δ 31.2, δ 29, δ 28.99, δ 28.91, δ 28.69, δ 28.65, δ 27.9, δ 25.2, δ 22, δ 13.9.

### Synthesis of Tetradecyl-D-glucaro-6,3-lactone monoester [tetradecyl(2R)-2-[(2S,3R,4S)-3,4-dihydroxy-5-oxo-tetrahydrofuran-2-yl]-2-hydroxy-acetate]

Above reaction was carried out similarly using tetradecanol (0.05205 moles) as alcohol. The product was crystallized from dichloromethane to obtain the desired tetradecyl-D-glucaro-6,3-lactone monoester (1.3 g).

¹H NMR (500 MHz, DMSO-d₆, TMS): δ 6.18-6.17 (d, 1H), δ 5.88-5.87 (d, 1H), δ 5.47-5.46 (d, 1H), δ 4.51-4.45 (m, 2H), δ 4.25-4.20 (t, 2H), δ 4.09-4.02 (d, 2H), δ 1.60-1.58 (d, 2H), δ 1.28 (bs, 22H), δ 0.87-8.85 (t, 3H).

¹³C NMR (500 MHz, DMSO-d₆, TMS): δ 176.3, δ 170.6, δ 80.5, δ 70.6, δ 70.1, δ 69.7, δ 64.7, δ 31.7, δ 29.5, δ 29.48, δ 29.42, δ 29.18, δ 29.15, δ 28.4, δ 25.7, δ 22.5, δ 14.4.

### Synthesis of Octyl-D-glucaro-6,3-lactone monoester [octyl(2R)-2-[(2S,3R,4S)-3,4-dihydroxy-5-oxo-tetrahydrofuran-2-yl]-2-hydroxy-acetate]

Above reaction was carried out similarly using octanol (0.26025 moles) as alcohol. The product was crystallized from heptane to obtain the desired octyl-D-glucaro-6,3-lactone monoester (6 g).

¹H NMR (500 MHz, DMSO-d₆, TMS): δ 6.17-6.16 (d, 1H), δ 5.87-5.86 (d, 1H), δ 5.46-5.45 (d, 1H), δ 4.50-4.44 (m, 2H), δ 4.25-4.20 (t, 2H), δ 4.10-4.02 (d, 2H), δ 1.61-1.56 (d, 2H), δ 1.25 (bs, 10H), δ 0.87-8.85 (t, 3H).

¹³C NMR (500 MHz, DMSO-d₆, TMS): δ 176.3, δ 170.6, δ 80.5, δ 70.6, δ 70.1, δ 69.7, δ 61.1, δ 31.7, δ 29.2, δ 29.1, δ 29, δ 28.4, δ 25.7, δ 22.5, δ 14.4.

### Synthesis of Hexadecyl-D-glucaro-6,3-lactone monoester [hexadecyl(2R)-2-[(2S,3R,4S)-3,4-dihydroxy-5-oxo-tetrahydrofuran-2-yl]-2-hydroxy-acetate]

Above reaction was carried out similarly using hexadecanol (0.26025 moles) as alcohol. The product was crystallized using suitable solvent to obtain desired hexadecyl-D-glucaro-6,3-lactone monoester (0.5 g).

¹H NMR (500 MHz, DMSO-d₆, TMS): δ 6.22-6.21 (d, 1H), δ 5.93-5.91 (d, 1H), δ 5.51-5.50 (d, 1H), δ 4.56-4.49 (m, 2H), δ 4.30-4.25 (t, 2H), δ 4.13-4.08 (d, 2H), δ 1.65-1.62 (d, 2H), δ 1.3 (s, 26H), δ 0.9-8.88 (t, 3H).

¹³C NMR (500 MHz, DMSO-d₆, TMS): δ 176.3, δ 170.6, δ 80.5, δ 70.6, δ 70.1, δ 69.7, δ 64.7, δ 31.7, δ 29.5, δ 29.48, δ 29.42, δ 29.18, δ 29.15, δ 28.4, δ 25.7, δ 22.5, δ 14.4.

1. A process for preparing D-glucaro-6,3-lactone monoester, the process comprising:
(A) reacting D-glucaro-6,3-lactone with at least one alcohol of general formula (I)
R₁—OH (I),
wherein
R₁ denotes an unsubstituted, linear or branched, C₁-C₂₀ alkyl, unsubstituted, linear or branched, C₂-C₂₀ alkenyl, unsubstituted or branched C₃-C₁₀ cycloalkyl, unsubstituted or branched C₃-C₁₀ cycloalkenyl, unsubstituted C₁-C₁₀ alkylene C₃-C₁₀ cycloalkyl, unsubstituted C₁-C₁₀ alkylene C₃-C₁₀ cycloalkenyl, —(CH₂CH₂O)ₙH, wherein n is an integer in a range of 1 to 20, or —(CH₂)ₙYR₂, wherein n is an integer in the range of 1-20;
Y denotes O or S; and
R₂ is independently selected from unsubstituted, linear or branched, C₁-C₂₀ alkyl, unsubstituted, linear or branched, C₂-C₂₀ alkenyl, unsubstituted or branched C₃-C₁₀ cycloalkyl, unsubstituted or branched C₃-C₁₀ cycloalkenyl, unsubstituted C₁-C₁₀ alkylene C₃-C₁₀ cycloalkyl, or unsubstituted C₁-C₁₀ alkylene C₃-C₁₀ cycloalkenyl, in a presence of at least one mineral acid, at least one Lewis acid, at least one carboxylic acid, or at least one sulfonic acid to obtain a compound of general formula (II) [chemical structure]
wherein
R₁ denotes an unsubstituted, linear or branched, C₁-C₂₀ alkyl, unsubstituted, linear or branched, C₂-C₂₀ alkenyl, unsubstituted or branched C₃-C₁₀ cycloalkyl, unsubstituted or branched C₃-C₁₀ cycloalkenyl, unsubstituted C₁-C₁₀ alkylene C₃-C₁₀ cycloalkyl, unsubstituted C₁-C₁₀ alkylene C₃-C₁₀ cycloalkenyl, —(CH₂CH₂O)ₙH, wherein n is an integer in a range of 1 to 20, or v-(CH₂)ₙYR₂, wherein n is an integer in a range of 1-20;
Y denotes O and S; and
R₂ denotes an unsubstituted, linear or branched, C₁-C₂₀ alkyl, unsubstituted, linear or branched, C₂-C₂₀ alkenyl, unsubstituted or branched C₃-C₁₀ cycloalkyl, unsubstituted or branched C₃-C₁₀ cycloalkenyl, unsubstituted C₁-C₁₀ alkylene C₃-C₁₀ cycloalkyl, or unsubstituted C₁-C₁₀ alkylene C₃-C₁₀ cycloalkenyl.

2. The process according to claim 1, characterized in that unsubstituted, linear C₁-C₂₀ alkyl is selected from the group consisting of methyl, ethyl, propyl, butyl, pentyl, hexyl, heptyl, octyl, nonyl, decyl, undecyl, dodecyl, tridecyl, tetradecyl, pentadecyl, octadecyl, heptadecyl, octadecyl, nonadecyl and eicosyl.

3. The process according to claim 1, characterized in that unsubstituted, branched C₁-C₂₀ alkyl is selected from the group consisting of iso-propyl, iso-butyl, t-butyl, iso-pentyl, neo-pentyl, 2-ethyl-hexyl, 2-propyl-heptyl, 2-butyl-octyl, 2-pentyl-nonyl, 2-hexyl-decyl, iso-hexyl, iso-heptyl, iso-octyl, iso-nonyl, iso-decyl, iso-dodecyl, iso-tetradecyl, iso-hexadecyl, iso-octadecyl and iso-eicosyl.

4. The process according to claim 1, characterized in that R₁ denotes an unsubstituted, linear or branched, C₁-C₂₀ alkyl, unsubstituted, linear or branched, C₂-C₂₀ alkenyl, unsubstituted C₃-C₁₀ cycloalkyl, unsubstituted C₃-C₁₀ cycloalkenyl, —(CH₂CH₂O)ₙH, wherein n is an integer in the range of 1 to 20, or —(CH₂)ₙYR₂, wherein n is an integer in the range of 1 to 20; Y is O; and R₂ denotes an unsubstituted, linear or branched, C₁-C₂₀ alkyl, unsubstituted, linear or branched, C₂-C₂₀ alkenyl, unsubstituted or branched C₃-C₁₀ cycloalkyl, or unsubstituted or branched C₃-C₁₀ cycloalkenyl.

5. The process according to claim 4, characterized in that R₁ denotes an unsubstituted, linear or branched, C₁-C₂₀ alkyl, unsubstituted, linear or branched, C₂-C₂₀ alkenyl, unsubstituted C₃-C₁₀ cycloalkyl, —(CH₂CH₂O)ₙH, wherein n is an integer in the range of 1 to 20, or —(CH₂)ₙYR₂, wherein n is an integer in the range of 1 to 20; Y is O; and R₂ denotes an unsubstituted, linear or branched, C₁-C₂₀ alkyl, unsubstituted, linear or branched, C₂-C₂₀ alkenyl, or unsubstituted or branched C₃-C₁₀ cycloalkyl.

6. The process according to claim 4, characterized in that R₁ denotes an unsubstituted, linear or branched, C₁-C₂₀ alkyl, unsubstituted C₃-C₁₀ cycloalkyl, —(CH₂CH₂O)ₙH, wherein n is an integer in the range of 1 to 20, —(CH₂)ₙYR₂, wherein n is an integer in the range of 1 to 20; Y is O; and R₂ denotes an unsubstituted, linear or branched, C₁-C₂₀ alkyl or unsubstituted or branched C₃-C₁₀ cycloalkyl.

7. The process according to claim 4, characterized in that R₁ denotes an unsubstituted, linear or branched, C₁-C₂₀ alkyl or —(CH₂CH₂O)ₙH, wherein n is an integer in the range of 1 to 20.

8. The process according to claim 1, characterized in that in step (A) the at least one mineral acid is selected from the group consisting of sulfuric acid, hydrochloric acid, phosphoric acid, perchloric acid, nitric acid, nitrous acid, sulphurous acid, chloric acid, chlorous acid, and hypochlorous acid.

9. The process according to claim 8, characterized in that in step (A) the at least one mineral acid is sulfuric acid.

10. The process according to claim 1, characterized in that in step (A) the at least one Lewis acid is a metal-containing compound selected from the group consisting of
a) AsX₃, GaX₃, BX₃, BX₃ (C₂H₅)₂O, BX₃.S(CH₃)₂, AlX₃, (C₂H₅)₂AlX, SbX₃, SbX₅, SnX₂, MgX₂, MgX₂.O(C₂H₅)₂, ZnX₂, BiX₃, FeX₂, TiX₂, TiX₄, NbX₅, NiX₂, CoX₂, HgX₂, whereby X in each case denotes F, Cl, Br, SO₃, CF₃—SO₃, CH₃—SO₃, or I,
b) BH₃, B(CH₃)₃, GaH₃, AlH₃, Al(acetate)(OH)₂, Al[OCH(CH₃)₂]₃, Al(OCH₃)₃, Al(OC₂H₅)₃, Al₂O₃, (CH₃)₃Al, Ti[OCH(CH₃)₂]₃C, Ti[OCH(CH₃)₂]₄, methylaluminum di-(2,6-di-tert-butyl-4-methylphenoxide), methylaluminum di-(4-brom-2,6-di-tert-butylphenoxide), LiClO₄,
c) Mg(acetate)₂, Zn(acetate)₂, Ni(acetate)₂, Ni(NO₃)₂, Co(acetate)₂, Co(NO₃)₂, Cu(acetate)₂, Cu(NO₃)₂, Li(acetate), Zr(acetylacetonate)₄, Si(acetate)₄, K(acetate), Na(acetate), Cs(acetate), Rb(acetate), Mn(acetate)₂, Fe(acetate)₂, Bi(acetate)₃, Sb(acetate)₃, Sr(acetate)₂, Sn(acetate)₂, Zr(acetate)₂, Ba(acetate)₂, Hg(acetate)₂, Ag(acetate), Tl(acetate)₃, Sc(fluoromethansulfonate)₃, Ln(fluoromethanesulfonate)₃, Ni(fluoromethanesulfonate)₂, Ni(tosylate)₂, Co(fluoromethanesulfonate)₂, Co(tosylate)₂, Cu(fluoromethanesulfonate)₂, and Cu(tosylate)₂.

11. The process according to claim 10, characterized in that the at least one Lewis acid is selected from the group consisting of BX₃, BX₃.S(CH₃)₂, AlX₃, SbX₃, and TiX₄, whereby X in each case denotes F, Cl, CF₃—SO₃, or CH₃—SO₃.

12. The process according to claim 10, characterized in that in step (A) the at least one Lewis acid is Al(CH₃—SO₃)₃.

13. The process according to claim 1, characterized in that in step (A) the at least one carboxylic acid is selected from the group consisting of trifluoroacetic acid, difluoroacetic acid, fluoroacetic acid, trichloroacetic acid, dichloroacetic acid, monochloroacetic, tribromoacetic acid, dibromoacetic acid, bromoacetic acid and iodoacetic acid.

14. The process according to claim 1, characterized in that in step (A) the at least one sulfonic acid is selected from the group consisting of methanesulfonic acid, ethanesulfonic acid, 1-propanesulfonic acid, 1-butanesulfonic acid, trifluoromethanesulfonic acid, benzenesulfonic acid, p-toluenesulfonic acid, p-xylene-2-sulfonic acid, naphathalene-1-sulfonic acid, and naphthalene-2-sulfonic acid.

15. The process according to claim 1, characterized in that step (A) is carried out in a presence of a molecular sieve.

16. The process according to claim 15, characterized in that in step (A) the molecular sieve has a pore diameter in a range of ≥0.1 Å to ≤10 Å.

17. The process according to claim 1, wherein step (A) is carried out in a presence of at least one polar aprotic solvent.

18. The process according to claim 17, characterized in that the at least one polar aprotic solvent is selected from the group consisting of ethers, lactones, carbonates, sulfones, N,N-dimethylformamide, N,N-dimethylacetamide, acetonitrile, dimethylsulfoxide, N-methyl-pyrrolidone and N-ethyl-pyrrolidone.

19. The process according to claim 18, characterized in that the ether is selected from the group consisting of methyl tert-butyl ether, dioxane, diethoxy methane, dimethoxy methane, tetrahydrofuran and tetrahydropyran.

20. The process according to claim 19, characterized in that the ether is dioxane.

21. The process according to claim 1, characterized in that in step (A) a molar ratio between the at least one alcohol of general formula (I) and D-glucaro-6,3-lactone is in a range of ≥0.1:1 to ≤5:1.

22. The process according to claim 1, characterized in that in step (A) the at least one acid is present in an amount in a range of ≥0.01 mol.-% to ≤20 mol.-% in relation to the D-glucaro-6,3-lactone.

23. The process according to claim 15, characterized in that in step (A) the molecular sieve is present in an amount in a range of ≥10 wt.-% to ≤40 wt.-% in relation to the D-glucaro-6,3-lactone.

24. The process according to claim 17, characterized in that a weight ratio between the at least one polar aprotic solvent and D-glucaro-6,3-lactone is in a range of ≥1:1 to ≤10:1.

25. The process according to claim 1, characterized in that in step (A) is carried out by stirring at a rotational speed in a range of ≥100 rpm to ≤500 rpm for a period in a range of ≥1 h to ≤10 h.

26. The process according to claim 1, characterized that step (A) is carried out at a temperature in a range of ≥30° C. to ≤90° C.

27. A compound of general formula (II); [chemical structure]
wherein
R₁ denotes unsubstituted, linear or branched, C₆-C₂₀ alkyl, unsubstituted, linear or branched, C₆-C₂₀ alkenyl, unsubstituted or branched C₃-C₁₀ cycloalkyl, unsubstituted or branched C₃-C₁₀ cycloalkenyl, unsubstituted C₁-C₁₀ alkylene C₃-C₁₀ cycloalkyl, unsubstituted C₁-C₁₀ alkylene C₃-C₁₀ cycloalkenyl, —(CH₂CH₂O)ₙH, wherein n is an integer in a range of 1 to 20, or —(CH₂)ₙYR₂, wherein n is an integer in a range of 1-20;
Y denotes O or S; and
R₂ denotes unsubstituted, linear or branched, C₁-C₂₀ alkyl, unsubstituted, linear or branched, C₂-C₂₀ alkenyl, unsubstituted or branched C₃-C₁₀ cycloalkyl, unsubstituted or branched C₃-C₁₀ cycloalkenyl, unsubstituted C₁-C₁₀ alkylene C₃-C₁₀ cycloalkyl, or unsubstituted C₁-C₁₀ alkylene C₃-C₁₀ cycloalkenyl.

28. The compound of formula (II) according to claim 27 is selected from the group consisting of
Decyl(2R)-2-[(2S,3R,4S)-3,4-dihydroxy-5-oxo-tetrahydrofuran-2-yl]-2-hydroxy-acetate,
Dodecyl(2R)-2-[(2S,3R,4S)-3,4-dihydroxy-5-oxo-tetrahydrofuran-2-yl]-2-hydroxy-acetate,
Tetradecyl(2R)-2-[(2S,3R,4S)-3,4-dihydroxy-5-oxo-tetrahydrofuran-2-yl]-2-hydroxy-acetate,
Octyl(2R)-2-[(2S,3R,4S)-3,4-dihydroxy-5-oxo-tetrahydrofuran-2-yl]-2-hydroxy-acetate, and
Hexadecyl(2R)-2-[(2S,3R,4S)-3,4-dihydroxy-5-oxo-tetrahydrofuran-2-yl]-2-hydroxy-acetate.
